(19) United States
(12) Patent Application Publication
(10) Pub. No.: US 2011/0318804 A1
(43) Pub. Date: Dec. 29, 2011

(54) PHOTOBIOREACTOR

(75) Inventors: Clemens Posten, Karlsruhe (DE); Anna Jacobi, Karlsruhe (DE); Christian Steinweg, Karlsruhe (DE); Florian Lehr, Ludwigshafen (DE); Rosa Rosello, Frankfurt am Main (DE)

(73) Assignee: KARLSRUHER INSTITUT FUER TECHNOLOGIE, Karlsruhe (DE)

(21) Appl. No.: 13/110,189

(22) Filed: May 18, 2011

(30) Foreign Application Priority Data
May 21, 2010 (DE) 10 2010 021 154.0

(51) Int. Cl. C12P 3/00; C12M 1/00

(52) U.S. Cl. 435/168; 435/292.1

(57) ABSTRACT

A bioreactor for cultivating phototrophic microorganism, includes a transparent upper plate and a lower plate, the upper plate being disposed above and spaced apart from the lower plate and defining therebetween a continuous cultivation volume having an inlet port and an outlet port, each of the plates including a plurality of parallel-facing deformations including peaks and troughs disposed in a regularly repeating geometric pattern. Further, a method of operating the bioreactor includes supplying a liquid culture of phototrophic microorganisms in the cultivation volume, supplying the microorganisms with nutrients, incubating the culture in daylight and harvesting at least one of the microorganisms and metabolites that have diffused into the culture medium are harvested.

### CROSS REFERENCE TO RELATED APPLICATIONS

This application claims priority to German Patent Application No. DE 10 2010 021 154, filed May 21, 2010.

### FIELD

The present invention relates to device and method for multiplying phototropic microorganisms. The device is hereinafter also referred to as a “photobioreactor”.

### BACKGROUND

Bioreactors, or fermentors, are devices for cultivating microorganisms under the best possible conditions so as to achieve optimum yield of cells or substances produced by cells. In conventional bioreactors, the decisive factors for the yield are primarily the input of nutrients for the organisms, the temperature and, possibly, the aeration.

When cultivating phototrophic organisms in a photobioreactor, there is an additional decisive factor, namely the input of light required as an energy source for phototrophic microorganisms to develop.

The production of microalgae is currently limited to a few thousand tons. There is a great interest in algae biomass because microalgae are a promising raw material for the production of fuels, such as biodiesel, biomethane or hydrogen. Algae biomass can also be used, for example, as a material to make medically active ingredients or as food or animal feed.

Conventional methods for obtaining algae biomass are typically carried out either in open systems or in closed photobioreactors. Closed photobioreactors have the advantages of allowing selective control of the reaction parameters, of minimizing contamination, and of achieving significantly higher productivities.

The production of biomass from phototrophic microorganisms requires light as an energy source, CO₂ or other organic molecules as a carbon source, and suitable nutrients in aqueous solution. The microorganisms used are initially grown and multiplied under sterile conditions. Then, the so-called inoculate is introduced into the photobioreactor along with the nutrient medium. Best possible multiplication and yield of biomass are achieved by controlling the introduction of gas and the pH-value, and by controlling the temperature to the optimum level.

In the process, the introduction of gas is often from above through the surface of the liquid culture medium, which is exposed to air. However, due to the small surface area and the low CO₂ concentration of the air, the gas input rate is, in this case, low. Alternatively, CO₂-enriched gas mixtures can be introduced by bubbles from below. The exchange surface and the CO₂ concentration gradient are thereby increased. However, this requires correspondingly high pumping energies, which has a negative effect on the energy balance.

German Patent Application DE 199 16 597 A1 describes a so-called “air-lift photobioreactor”. This type of tube reactor achieves good mixing by introducing gas vertically from below and is capable of inducing what is known as a “flashing-light effect” by increasing the surface area using extensions and internals. The flashing-light effect is an effect where increased growth rates can be achieved by rapidly changing light intensities (bright-dark cycles). These cycles are due to the reactor geometry and the introduction of gas, as a result of which the algae are subjected to turbulent flow, and thereby caused to rapidly fluctuate between well-illuminated and shaded sites.

US Patent Application No. 2009/0305389 A1 describes photobioreactors in which CO₂ is introduced through membranes. In contrast to the present invention, this reactor is a film reactor. The membranes are not integrated into the surface of the reactor, but implemented in the suspension as rigid membrane tubes.

German Patent Application DE 10 2008 031 769 A1 proposes a flat structure in which the growth chambers are separated from each other and are supplied with water and CO₂ through inlet and outlet chambers. Consequently, this reactor is not a flow-through type reactor. Mixing is preferably accomplished by gas-bubble-induced cylindrical rotation of the liquid culture medium. These photobioreactors are configured in such a manner that the individual modules can be arranged in series or parallel. The input of light energy is ensured solely by the fact that the materials used are preferably transparent and may optionally be made from wavelength-shifting materials. There is no mention of this design increasing the surface area for refractive light input.

A fundamental problem in the cultivation of phototrophic microorganisms is the poor tolerance of most species to high light intensities. Most microalgae exhibit saturation effects at light intensities significantly lower than the maximum daylight intensity of about 200 Watt/m². On the other hand, it is desired to use the maximum amount of light possible so as to achieve high photon conversion efficiencies. In the bioreactors published heretofore, the entering light field is neither equalized nor diluted. The intensities with which the microorganisms are illuminated exhibit undefined high gradients, which are not specifically measured. Therefore, such reactors are unable to yield maximum productivity.

In both open and closed systems, the algae biomass is typically moved by suitable devices (paddle-wheels, pumps, air streams in the reactor, reactor design) to prevent it from settling, and to achieve improved illumination of the algae. This requires considerable energy input into the systems, which worsens the energy balance.

Furthermore, it is common to use complex supporting structures to stabilize the reactors in the vertical position, for example, against wind pressure. An alternative is to use a greenhouse which, however, causes higher costs itself.

In order to achieve economic operation, also with a view to later processing, the concentration of microalgae in the medium, in addition to the productivity per surface area, is a decisive factor. Until now, closed reactors have been operated at concentrations of no more than 2 g/l, inter alia, to prevent self-shadowing of the culture in high cell density conditions.

In spite of the new technological approaches, the prior art has so far been unable to reduce the cost of photobioreactors to around 25 [symbol] /m², which is predicted in studies to allow economic energetic use of microalgae. Moreover, the amount of auxiliary energy required for introducing gas and mixing is far too high. Typical values are above 5 W/m², which alone would consume the expected energy gain from the microalgae. Therefore, the reactor systems that have been published so far are not suitable for use in energy applications. Further problems arise when the intention is to produce hydrogen.

### SUMMARY

In an embodiment, the present invention provides bioreactor for cultivating phototrophic microorganism includes a transparent upper plate and a lower plate, the upper plate being disposed above and spaced apart from the lower plate and defining therebetween a continuous cultivation volume having an inlet port and an outlet port, each of the plates including a plurality of parallel-facing deformations including peaks and troughs disposed in a regularly repeating geometric pattern.

In another embodiment, the invention provides a method of operating the bioreactor that includes supplying a liquid culture of phototrophic microorganisms in the cultivation volume, supplying the microorganisms with nutrients, incubating the culture in daylight and harvesting at least one of the microorganisms and metabolites that have diffused into the culture medium are harvested.

### BRIEF DESCRIPTION OF THE DRAWINGS

Exemplary embodiments of the present invention are described in more detail below with reference to the drawings, in which:

FIG. 1 is a schematic view of a bioreactor according to an embodiment of the present invention;

FIG. 2 is a schematic cross-sectional view through the bioreactor.

### DETAILED DESCRIPTION

In an embodiment, the present invention provides a bioreactor for cultivating phototropic microorganisms and a method for operating the same. In another embodiment, the invention provides the use of the bioreactor for producing fuels. In this regard, the intention is for the bioreactor to ensure an optimized refractive light input so as to enable a culture of high cell density. The pumping energy input required for circulation, introduction of gas, and mixing is preferably kept as low as possible. Due to its design, the bioreactor is inexpensive to manufacture and operate, which allows an economically reasonable production of biomass and fuels.

A bioreactor in accordance with one embodiment of the invention is used for cultivating phototrophic microorganisms. The bioreactor design includes two plates, namely an upper transparent plate and a lower plate. Both plates are disposed one above the other in substantially parallel relationship, so that a continuous cultivation volume is provided between the plates. The small distance between the two plates corresponds to the height of the cultivation volume and is preferably from 0.1 mm to 40 mm, particularly preferably from 0.1 mm to 10 mm. Due to the small layer thickness, the probability of the organisms shadowing each other is lower than with greater layer thicknesses.

In order for the distance between the two plates to be constant across the entire area of the bioreactor, spacers may optionally be placed between the upper and lower plates to prevent the upper plate from sagging because of its properties, which would reduce the distance between the two plates. This sagging is dependent on the size and thickness of the plate, and above all on the materials used. The number and distribution of the spacers should be adjusted according to these factors.

The shape of the two plates is characterized by a plurality of peaks and troughs which are formed in both the upper and lower plates, so that the cultivation volume has a uniform layer thickness across the area. The peaks and troughs extend parallel to each other across the entire width of the reactor in a regularly repeating geometric pattern. This geometry gives the bioreactor and the cultivation volume preferably a wave or zigzag shape. Unlike the flat-plate reactors known in the art, which have a substantially flat cultivation surface, this wave or zigzag shape allows for dilution of the entering light. In particular when the bioreactors are operated in sunlight, the intensity of which is far above the saturation intensity of the phototrophic organisms, it is thereby possible to illuminate a larger area with a more moderate light intensity.

The bioreactor includes at least one inlet port and one outlet port, which allow control of essential functions. Firstly, the bioreactor can be charged with a culture; secondly, the culture can be circulated within the cultivation volume by suitable means; and ultimately, the culture can be discharged or harvested.

In an embodiment, the inlet and outlet ports are disposed in such a way that the flow of the culture within the bioreactor is in a direction parallel along the peaks and troughs. This direction of flow has the advantage that the flow resistance is lower than would be the case if the liquid medium flowed across the peaks and troughs. This allows the reactor to be operated with less pumping energy. Due to the design of the bioreactor, excessive pump pressure may cause deformation thereof, which is to be avoided.

A plurality of such bioreactors can be connected to each other via the inlet and outlet ports, so that a larger cultivation volume is created. The bioreactors may be connected in series or in parallel.

In an embodiment, the overall height of the peaks and troughs is from 0.5 cm to 30 cm, particularly preferably from 2 cm to 10 cm.

In another embodiment of the bioreactor according to the present invention, the base area of the bioreactor is from 0.5 m² to 50 m², preferably from 1 m² to 25 m². In this context, the base area is not the same as the surface area of the cultivation volume, which is larger because of the peaks and troughs.

The ratio of the base area of the bioreactor to the surface area of the cultivation volume is preferably in the range from 1:2 to 1:10.

In an embodiment of the reactor of the present invention, the number of peaks and troughs per meter of reactor width is from 10 to 100.

The transparent upper plate of the bioreactor is preferably provided with an IR-reflective coating. Compared to light in the visible wavelength range, which is used for photosynthesis of the phototrophic organisms, infrared light is to be considered first and foremost as thermal radiation which, when the reactor is operated in sunlight, may strongly heat up the reactor at certain times of the day. The IR-reflective coating allows a large part of the thermal radiation to be removed.

In an embodiment of the bioreactor, in order to optimize the illumination of the cultivation volume, the light transmitted through the cultivation volume can be reflected back into the cultivation volume by a light-reflective coating on the lower plate. In this manner, a large part of the transmitted light is also made available to the phototrophic organisms that are illuminated by light reflection from the bottom of the reactor. Radiation loss is minimized by the light-reflective coating.

Another decisive factor for the efficiency of a photobioreactor is an optimum supply of CO₂. Preferably, the introduction of gas is through permeable membranes, which are integrated into the lower plate. It is an advantage that the energy input required for introducing gas is minimized by using gassing membranes where the transition from the gaseous to the dissolved phase already occurs in the material of the membrane. The gassing membranes may vary in shape. For example, the membrane may be tubular and extend through the culture medium (for example along the troughs), and may be attached to the lower plate. Alternatively, the membrane may be a flat membrane that makes portions of the lower plate permeable for passage of CO₂ therethrough. In this case, it is advantageous for the bioreactor to be hermetically sealed at the bottom by an additional lower cover. Thus, a gas stream, which may be enriched with CO₂, can pass between the lower cover and the lower plate, and in the process, CO₂ can diffuse through the membrane into the culture. The introduction of gas through membranes is advantageous for minimizing the use of hydraulic and pneumatic auxiliary energy. The formation of gas is used for mass transfer, so that the pumping energy requirement is minimized. Ideally, the supply with CO₂ is accomplished by a higher CO₂ partial pressure, which is achieved by CO₂ enrichment of the gas supply.

In another embodiment of the bioreactor of the present invention, the lower plate may be equipped with sensors; i.e., the sensors may be integrated into the lower plate and used to monitor various cultivation parameters. Examples of such cultivation parameters are the dissolved concentrations of O₂ and CO₂, the pH-value, the optical density and, above all, the temperature. The monitoring of cultivation parameters serves for the control of the bioreactor, making it possible to provide optimum culture conditions. In particular, it is possible for the sensor-controlled bioreactor to operate autonomously, which allows for cost-effective distributed operation.

Optionally, the bioreactor of the present invention is equipped with an upper cover, which may serve to protect against the effects of extreme weather conditions, such as hail impact or the like.

Moreover, this upper cover can be used for hermetically sealing the bioreactor. For example, if the bioreactor is used for producing hydrogen from microalgae, the hydrogen produced may diffuse through the material of the upper plate. In this case, the upper plate is preferably not made from glass or other material that is impermeable to hydrogen, whereas the upper cover is preferably made from glass or other hydrogen-impermeable material, so that the gas will be collected between the upper cover and the upper plate. Due to its size and properties, hydrogen can diffuse through most polymeric materials.

In an embodiment, the present invention also relates to a method for operating a bioreactor, including the following steps:
a) providing a bioreactor according to the present invention and as described in the preceding paragraphs;
b) filling the cultivation volume of the bioreactor with a liquid culture containing phototrophic microorganisms;
c) supplying the microorganisms with nutrients, which are distributed in the culture medium mainly by diffusion and convection;
d) incubating the culture in daylight;
e) harvesting the microorganisms if the intention is to use the cells as biomass or to extract substances contained in the cells, or separating the metabolites (e.g., hydrogen) that have diffused into the medium.

The culture is operated as a static culture (batch culture) or continuous culture.

Preferably, the bioreactor is operated in a horizontal orientation; i.e., substantially parallel to the surface of the earth.

A batch culture is understood to be a discontinuous method of cultivation, where the bioreactor is charged with a culture once, and the culture remains therein until it is harvested. Here too, the culture needs to be circulated and supplied with essential substances, such as CO₂.

A continuous culture is operated in continuous regime. Growth, multiplication and harvest of the culture and metabolites, respectively, are carried out continuously. This also means that the phototrophic microorganisms are continuously supplied with nutrients.

In an embodiment of the method, in step c), the culture is supplied with carbon dioxide through gas-permeable membranes. In the method of the present invention, sufficient supply with nutrients can be ensured mainly by diffusion and slight convection (thermal convection and slight circulation of the culture medium) due to the small layer thickness of the bioreactor. Excessive pumping energies should be avoided because the reactor may inflate because of its design.

When the method of the present invention is used for producing gaseous metabolites, the gases produced can be separated in step e), preferably by suitable membranes. To this end, for example, part of the reactor surface may be replaced with membrane materials. This design would allow the gases to be separated through the upper or lower plate. However, this requires an additional cover above and underneath the two reactor plates, respectively, because otherwise the gases would escape.

Charging the reactor with CO₂ during the production of gaseous metabolites may cause the formation of gas mixtures. However, this problem may be solved by CO₂ separation means known to those skilled in the art.

The bioreactor presented here, and the method for operating the same, are particularly suitable for producing phototrophic microorganisms in an autonomous and economical manner. The closed design of such a bioreactor allows, for example, operation in distributed areas in arid, sunny climates. The bioreactors of the present invention may be used for producing hydrogen from suitable phototrophic microorganisms.

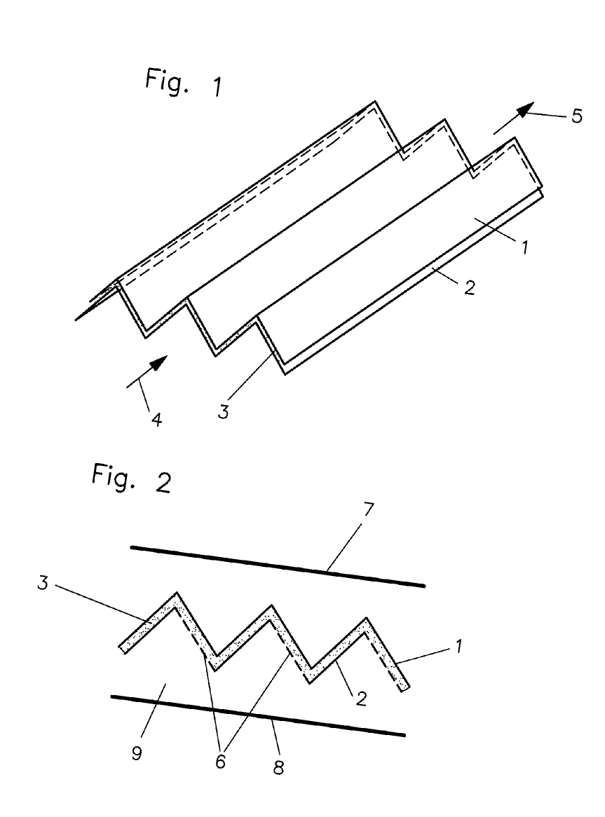

FIG. 1 illustrates in schematic form the zigzag design of the bioreactor. Cultivation volume 3 is located between upper plate 1 and lower plate 2. This cultivation volume 3 contains the culture of phototrophic microorganisms. Light enters refractively from above. It can be seen that the surface area of the culture is greater than the total area occupied by the reactor. Due to the zigzag structure, the light is always incident at an angle on the surface of the culture. This reduces the radiation intensity and increases the total surface area being irradiated.

Further, the bioreactor shown in FIG. 1 is provided with an inlet port 4 and an outlet port 5. The preferred direction of flow in cultivation volume 3 is parallel to the peaks and troughs.

FIG. 2 shows, in a cross-sectional view, the bioreactor of FIG. 1, which is here additionally provided with an upper cover plate 7 and a lower cover plate 8. This view illustrates how CO₂ is supplied through a membrane 6 from the lower gas space 9 into cultivation volume 3. This method of introducing CO₂ is mainly based on diffusion and, therefore, does not require high levels of positive pressure.

While the invention has been particularly shown and described with reference to preferred embodiments thereof, it will be understood by those skilled in the art that various changes in form and details may be made therein without departing from the spirit and scope of the invention.

### LIST OF REFERENCE NUMERALS

1 upper plate

2 lower plate

3 cultivation volume

4 inlet port

5 outlet port

6 membrane

7 upper cover plate

8 lower cover plate

9 gas space for the supply of CO₂

1-13. (canceled)

14. A bioreactor for cultivating phototrophic microorganism comprising:
a transparent upper plate and a lower plate, the upper plate being disposed above and spaced apart from the lower plate and defining therebetween a continuous cultivation volume having an inlet port and an outlet port, each of the plates including a plurality of parallel-facing deformations including peaks and troughs disposed in a regularly repeating geometric pattern.

15. The bioreactor as recited in claim 14, wherein the plurality of deformations have at least one of a wave and a zigzag shape.

16. The bioreactor as recited in claim 14, wherein an overall height of the peaks and troughs is in a range from 1 cm to 20 cm.

17. The bioreactor as recited in claim 14, wherein a distance between the upper plate and the lower plate is in a range from 0.1 mm to 40 min.

18. The bioreactor as recited in claim 14, wherein the upper plate includes an IR-reflective coating,

19. The bioreactor as recited in claim 14, wherein the lower plate includes a light-reflective coating.

20. The bioreactor as recited in claim 1, wherein the lower plate includes at least one of gas-permeable membranes, membrane strips and membrane tubes,

21. An array of a plurality of bioreactors, each bioreactor comprising:
a transparent upper plate and a lower plate, the upper plate being disposed above and spaced apart from the lower plate and defining therebetween a continuous cultivation volume having an inlet port and an outlet port, each of the plates including a plurality of parallel-facing deformations including peaks and troughs disposed in a regularly repeating geometric pattern.

22. A method for operating a bioreactor comprising:
providing a bioreactor including a transparent upper plate and a lower plate, the upper plate being disposed above and spaced apart from the lower plate and defining therebetween a continuous cultivation volume having an inlet port and an outlet port, each of the plates including a plurality of parallel-facing deformations including peaks and troughs disposed in a regularly repeating geometric pattern;
providing a liquid culture of phototrophic microorganisms in the cultivation volume of the bioreactor;
supplying the microorganisms with nutrients by diffusion and convection within a culture medium of the culture;
incubating the culture in daylight; and
harvesting at least one of the microorganisms and metabolites that have diffused into the culture medium.

23. The method as recited in claim 22, wherein the culture is operated as one of a static culture (batch culture) and a continuous culture.

24. The method as recited in claim 22, wherein the supplying the microorganisms with nutrients includes supplying carbon dioxide through gas-permeable membranes.

25. The method as recited in claim 22, wherein the harvesting includes transporting gaseous metabolites through a surface of the liquid, and separating the gaseous metabolites by at least one of membranes and gas-permeable reactor materials.

26. A method of producing hydrogen comprising:
providing a bioreactor including a transparent: upper plate and a lower plate, the upper plate being disposed above and spaced apart from the lower plate and defining therebetween a continuous cultivation volume having an inlet port and an outlet port, each of the plates including a plurality of parallel-facing deformations including peaks and troughs disposed in a regularly repeating geometric pattern; and
producing hydrogen from phototrophic microorganisms using the bioreactor.
